(19) United States
(12) Patent Application Publication
(10) Pub. No.: US 2016/0333505 A1
(43) Pub. Date: Nov. 17, 2016

(54) MICROCLIMATE CONTROLLING FABRICS AND METHODS

(71) Applicant: Precision Fabrics Group, Inc., Greensboro, NC (US)

(72) Inventors: W. Allen Leonard, Greensboro, NC (US); Neil Blanton, Oak Ridge, NC (US); Terry G. Montgomery, Greensboro, NC (US); Kavita Mathur, Cary, NC (US)

(21) Appl. No.: 15/218,603

(22) Filed: Jul. 25, 2016

Related U.S. Application Data
(63) Continuation of application No. 13/534,468, filed on Jun. 27, 2012.
(60) Provisional application No. 61/505,284, filed on Jul. 7, 2011.

(51) Int. Cl. D03D 15/00; A61F 13/538; A41D 13/12; D03D 13/00; A61F 5/30

(52) U.S. Cl. CPC D03D 15/00; D03D 13/008; D03D 15/0083; A61F 5/30; A41D 13/1236; A61F 13/538; D10B 2503/06; A61F 2013/1552

(57) ABSTRACT

A fabric comprises a woven fabric having continuous filament warp yarns and continuous filament filling yarns, wherein the warp yarns are about 30 denier to 100 denier, and the filling yarns are about 30 denier to 100 denier, wherein the warp yarns and filling yarns are woven together in a plain weave to provide a pill resistant fabric having identical surfaces on both sides thereof, and wherein each surface has a coefficient of friction that does not exceed 0.35 under both dry and wet surface conditions. The fabric has a wicking rate of between about 1.00 and 1.75 inches per minute, and has a percent dryness after 45 minutes of 90%.

### RELATED APPLICATION

This application claims the benefit of and priority to U.S. Provisional Patent Application No. 61/505,284 filed Jul. 7, 2011, the disclosure of which is incorporated herein by reference as if set forth in its entirety.

### FIELD OF THE INVENTION

The present invention relates generally to fabrics and, more particularly, to bedding fabrics and clothing fabrics.

### BACKGROUND

Skin irritations and wounds are a growing problem in the United States and around the world. The standard treatments for chronic skin problems involve the routine application of antibacterial ointments to reduce the potential for infections. Other treatments include the avoidance of clothing made of irritating wool or natural fibers and the use of non-aggressive detergents on clothing and bedding fabrics.

Skin irritations and wounds typically are exacerbated by exposure to excessive moisture, and to shear forces caused by friction with clothing and bedding fabrics. For bedridden persons, moisture and friction at the skin-fabric interface are often associated with the feeling of discomfort (for example, when fabric sticks to the skin). However, even more critical for bedridden persons, moisture, friction, and shear are proximate causes of mechanical skin irritations, trauma, and pressure ulcers. Pressure, heat, friction, and shear, in combination with moisture, as illustrated in FIG. 1, may accelerate and promote the formation of pressure ulcers. Once the skin has been damaged by one or a combination of these factors, the development of pressure-related damage may be more likely to take place.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

In view of the above, a fabric, according to some embodiments of the present invention, comprises a woven fabric having continuous filament warp yarns and continuous filament filling yarns, wherein the warp yarns are about 30 denier to 100 denier, and the filling yarns are about 30 denier to 100 denier, wherein the warp yarns and filling yarns are woven together in a plain weave to provide a pill resistant fabric having identical surfaces on both sides thereof, and wherein each surface has a kinetic coefficient of friction (COF) that does not exceed 0.35 under both dry and wet surface conditions. In addition, the fabric has a wicking rate of between about 1.00 and 1.75 inches per minute, and has a percent (%) dryness after 45 minutes of about 90%.

In some embodiments of the present invention, one of the warp or filling yarns is 100% continuous filament polymer having round filament cross sections and makes up at least 40% by weight of the fabric. The other of the warp or filling yarns is continuous filament polymer having non-round filament cross sections and makes up the remainder of the weight of the fabric.

In some embodiments of the present invention, one of the warp or filling yarns comprises 100% continuous filament nylon having round filament cross sections and making up at least 40% by weight of the fabric, and the other of the warp or filling yarns comprises continuous filament polyester having non-round filament cross sections or nylon having non-round filament cross sections and making up the remainder of the weight of the fabric.

In some embodiments of the present invention, the warp yarns are 100% nylon, such as 70 denier, 48 filament, textured, continuous filament nylon yarns.

In some embodiments of the present invention, the filling yarns are 100% polyester, such as 75 denier, 36 filament, continuous filament textured polyester yarns.

In some embodiments of the present invention, the warp and filling yarns are 70 denier, 200 filament, textured, continuous filament polyester yarns.

In some embodiments of the present invention, the warp or filling yarns with non-round filament cross sections are configured such that adjacent filaments form wicking channels. Exemplary non-round filament cross sections include, but are not limited to, star shaped cross sections and clover leaf cross sections.

In some embodiments of the present invention, an antimicrobial substance is topically applied to the fabric. In some embodiments of the present invention a soil-release is topically applied to the fabric.

Fabric according to embodiments of the present invention has zero percent (0%) weight loss after one hundred (100) launderings in accordance with the AATCC 61 test method. In addition, fabric according to embodiments of the present invention has an average Kawabata geometric roughness of less than about 1.2 microns.

According to some embodiments of the present invention, a fabric comprises a woven fabric having continuous filament warp yarns and continuous filament filling yarns. The warp yarns are about 30 denier to 100 denier, and the filling yarns are about 30 denier to 100 denier, and the warp yarns and filling yarns are woven together in a plain weave to provide a pill resistant fabric having identical surfaces on both sides thereof. Each surface has a kinetic coefficient of friction that does not exceed 0.30 under both dry and wet surface conditions. In addition, the fabric has zero percent (0%) weight loss after one hundred (100) launderings in accordance with the AATCC 61 test method. The fabric also has a wicking rate of between about 1.00 and 1.75 inches per minute and a percent (%) dryness after 45 minutes of 90%. In some embodiments of the present invention, each surface of the fabric has a kinetic coefficient of friction that does not exceed 0.30 under dry conditions and a kinetic coefficient of friction that does not exceed 0.22 under wet conditions.

Fabrics according to embodiments of the present invention can be utilized to manufacture various articles utilized in medical facilities and are not limited to the manufacture of sheets (i.e., bottom sheets and top sheets) and hospital gowns. For example, fabrics according to embodiments of the present invention can be utilized to manufacture pillow cases, underpads, masks, cubicle, surgical caps, uniforms, etc.

Fabrics according to embodiments of the present invention when utilized as bed sheets and hospital gowns, can reduce the number of patient pressure wounds developed within a medical facility. For example, the average number of pressure wounds developed by patients in a medical facility per one thousand (1,000) patient days can be reduced by more than half (e.g., 50%) for patients occupying beds fitted with the bed sheets and wearing the hospital gowns, as compared with patients occupying beds fitted with conventional polyester/cotton bed sheets and wearing conventional polyester/cotton hospital gowns. Moreover, the average number of pressure wounds resolved per one thousand (1,000) patient days can be increased by at least about twenty nine percent (29%) for patients occupying beds fitted with the bed sheets and wearing the hospital gowns, as compared with patients occupying beds fitted with conventional polyester/cotton bed sheets and wearing conventional polyester/cotton hospital gowns.

According to some embodiments of the present invention, a method of reducing the number of pressure wounds developed by a patient within a medical facility includes fitting a bed within the medical facility with clean sheets, wherein each sheet comprises a woven fabric having warp yarns and filling yarns woven together in a plain weave to provide a pill resistant fabric having identical surfaces on both sides thereof, and wherein each surface has a kinetic coefficient of friction that does not exceed at least 0.20 under both dry and wet surface conditions, then occupying the bed with the patient. In some embodiments, each surface of the fabric has a kinetic coefficient of friction that does not exceed at least 0.30 under both dry and wet surface conditions. In some embodiments, each surface of the fabric has a kinetic coefficient of friction that does not exceed at least 0.35 under both dry and wet surface conditions.

According to some embodiments of the present invention, a method of reducing the number of pressure wounds developed by a patient within a medical facility includes dressing the patient with a clean hospital gown, wherein the hospital gown comprises a woven fabric having warp yarns and filling yarns woven together in a plain weave to provide a pill resistant fabric having identical surfaces on both sides thereof, and wherein each surface has a kinetic coefficient of friction that does not exceed at least 0.20 under both dry and wet surface conditions; fitting a bed with clean sheets, wherein each sheet. comprises the woven fabric; and then occupying the bed with the patient. In some embodiments, each surface of the fabric has a kinetic coefficient of friction that does not exceed at least 0.30 under both dry and wet surface conditions. In some embodiments, each surface of the fabric has a kinetic coefficient of friction that does not exceed at least 0.35 under both dry and wet surface conditions.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate some exemplary embodiments. The drawings and description together serve to fully explain the exemplary embodiments.

FIG. 1 is a schematic illustration of a person on a support surface and illustrating the presence of pressure, heat, friction and shear at the skin-fabric interface that, in combination with moisture, may accelerate and promote the formation of pressure ulcers.

FIG. 2 is a graph of a comparison of particle and lint generation between fabrics according to embodiments of the present invention and conventional cotton-blend hospital top and bottom sheets.

FIG. 3 is an illustration of an exemplary bed covered with sheet(s) made from fabric according to some embodiments of the present invention, and exemplary pillows covered with pillowcases made from fabric according to some embodiments of the present invention.

FIG. 4 is a photomicrograph of a yarn with filaments having star-shaped fiber cross sections according to some embodiments of the present invention.

FIG. 5 is a photomicrograph of a yarn with filaments having cloverleaf-shaped fiber cross sections according to some embodiments of the present invention.

FIG. 6A is a photomicrograph of a fabric according to some embodiments of the present invention.

FIG. 6B is a photomicrograph of the fabric of FIG. 6A after being laundered one hundred seventy five (175) times.

FIG. 7A is a photomicrograph of a conventional cotton/polyester hospital bedding fabric.

FIG. 7B is a photomicrograph of the cotton/polyester fabric of FIG. 7A after being laundered one hundred seventy five (175) times.

FIG. 8 is a graph illustrating fiber loss for a fabric according to some embodiments of the present invention after extended laundering and for a conventional polyester/cotton fabric currently used in hospital sheets after extended laundering.

FIG. 9 is a graph illustrating that hospital bedding and gown fabrics according to embodiments of the present invention decreased the average length of stay of a patient within a hospital.

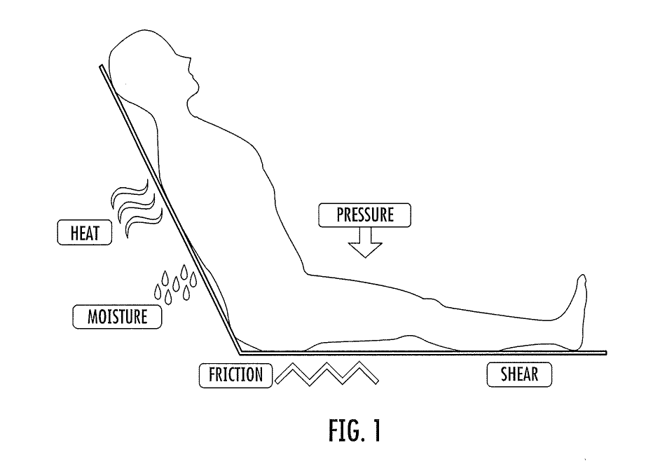

### DETAILED DESCRIPTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element, or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the fabric in use or operation in addition to the orientation depicted in the figures. For example, if an illustrated embodiment in the figures is inverted, elements described as “under” or “beneath” other elements or features would then be oriented “over” the other elements or features. Thus, the exemplary term “under” can encompass both an orientation of “over” and “under”. The embodiment may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

The term “bed”, as used herein, refers to any device or apparatus having a support surface upon which or within which a patient sleeps, rests, or stays, for example, when in a medical facility.

The term “clean”, as used herein, refers to a fabric that has been laundered.

The term “coefficient of friction” (COF), as used herein, is the ratio of the frictional force to the force acting perpendicular (e.g., gravitational force) to two surfaces in contact. COF is a measure of the difficulty with which the surface of one material will slide over an other material. The static or starting coefficient of friction is related to the force required to begin movement of one of the material. The kinetic or sliding coefficient of friction is related to the force measured during movement.

The terms “filament” and “fiber”, as used herein, are interchangeable. Yarns utilized in producing fabrics according to embodiments of the present invention are formed from a plurality of continuous filaments/fibers.

The term “fitting”, as used herein with respect to beds, refers to making a bed with a sheet that conforms to the shape of a mattress or other patient support surface (i.e., a bottom sheet). Also, top sheets may be fitted to a bed.

The term “non-treated woven fabric”, as used herein, refers to a woven fabric that has not been treated with an antimicrobial substance.

The term “medical facility”, as used herein, refers to any location at which health care is provided to patients and includes, but is not limited to, hospitals, clinics, and the like.

The term “microclimate”, as used herein, refers to skin moisture and temperature levels at a skin-fabric interface.

The term “pathogen”, as used herein, refers to any infectious biological agent such as a virus, bacteria, prion, or fungus that may cause disease to its host.

Fabrics, according to embodiments of the present invention, may have various uses including, but not limited to, bedding, blankets, pillow encasements, underpads, mattress encasements, gowns, bandages for wound care, curtains and draperies, patient moving/lifting devices, patient support systems, wheel chair covers and encasements, booties, sleep sacks, etc.

Applicants have unexpectedly discovered that to help protect a person's skin and prevent damage from occurring when the person is confined to a bed or other support surface, and thus make it more difficult for skin and wound issues to develop, a fairly narrow range of surface temperatures and humidity, referred to herein as the “microclimate”, should be maintained in the air immediately surrounding the body. Higher temperatures from support surfaces and fabrics associated therewith (e.g., bedding fabrics, patient gowns, etc.) cause and/or increase sweating. Increased moisture leads to maceration of the skin. In turn, maceration may reduce skin strength up to 96% and may increase the coefficient of friction of a support surface. Friction and tangential pressure quickly leads to shear stress on the skin of a person, which can break down the skin. High shear stress, in turn, may lead to greater injury. Shear stress may also significantly reduce the time needed to develop a pressure sore.

Fabric according to embodiments of the present invention effectively manages moisture, friction and shear between the skin and the fabric on a support surface (e.g., a bed, etc.), and thereby can reduce the development of new pressure wounds. Fabrics, according to embodiments of the present invention, are formed with a woven fabric having warp yarns and filling yarns woven to provide an identically smooth fabric surface on both sides thereof. In some embodiments, one of the warp or filling yarns is at least 40% by weight of the fabric of a continuous filament polymer, such as nylon or polyester. For example, one of the warp or filling yarns is at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, by weight of the fabric of continuous filament nylon or polyester. The other of the warp or filling yarns is 60% or less by weight of the fabric of continuous filament polyester or nylon having non-round filament cross sections. For example, the other of the warp or filling yarns may be 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight of the fabric of continuous filament polyester or nylon having non-round filament cross sections. In some embodiments, the warp yarns may be 100% nylon, and the filling yarns may be polyester or nylon.

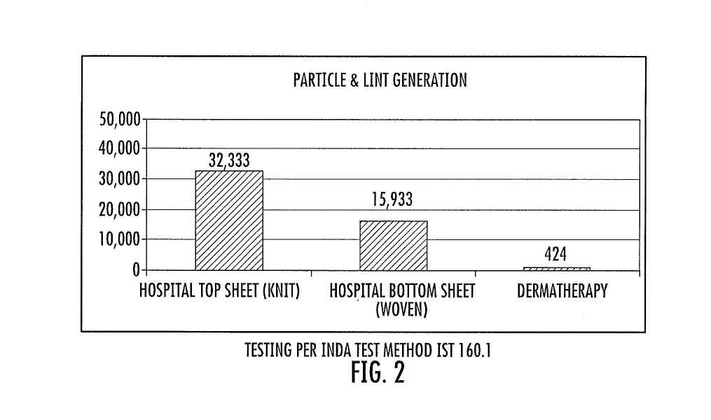

In one embodiment, the fabric is woven as a plain weave. Yarns are woven into fabric constructions that have 80% to 100% coverage. The warp yarn may be, for example, a 40 denier, 34 filament, five twist per inch, continuous filament nylon 6-6 yarn, and the filling yarn may be a 75 denier, 48 filament, continuous filament textured polyester. In another embodiment, the warp yarn is, for example, a 70 denier, 48 filament, continuous filament, textured nylon, and the filling yarn is, for example, a 75 denier, 36 filament, continuous filament, textured polyester. Continuous filament yarns are utilized because those yarns do not have short fibers extending beyond the fabric's planar surface, thereby decreasing irritation to sensitive skin. Also, fibers extending beyond a fabric's planar surface, as found in conventional cotton-blend hospital fabrics, can loosen, shed or break off and thereby contribute to an increased coefficient of friction (COF), both static and kinetic, at the skin-fabric interface. Moreover, such fiber particles can then contaminate open wounds or be inhaled or ingested by patients, caretakers, and housekeeping staff. This difference is readily seen in FIG. 2, where the relative particle and lint generation of fabrics according to embodiments of the present invention and conventional cotton-blend hospital linens are measured and compared.

The data shown in FIG. 2 indicates reductions in particle generation of 98.7% when fabrics according to embodiments of the present invention are compared with conventional cotton fitted sheets and 97.0% when compared with conventional cotton top sheets. Thus, the smooth, identical surfaces of fabric according to embodiments of the present invention mitigate the potential for friction and shear, as well as contamination from particles.

In other embodiments of the present invention, warp yarns of about 30 denier to 100 denier, and filling yarns of about 30 denier to 100 denier may be used. For example, warp and filling yarns may have any mass density within the range of 30 denier to 100 denier (e.g., 31 denier, 32 denier, 33 denier, 34 denier, 35 denier, 36 denier, 37 denier, 38 denier, 39 denier, 40 denier, 41 denier, 42 denier, 43 denier, 44 denier, 45 denier, 46 denier, 47 denier, 48 denier, 49 denier, 50 denier, 51 denier, 52 denier, 53 denier, 54 denier, 55 denier, 56 denier, 57 denier, 58 denier, 59 denier, 60 denier, 61 denier, 62 denier, 63 denier, 64 denier, 65 denier, 66 denier, 67 denier, 68 denier, 69 denier, 70 denier, 71 denier, 72 denier, 73 denier, 74 denier, 75 denier, 76 denier, 77 denier, 78 denier, 79 denier, 80 denier, 81 denier, 82 denier, 83 denier, 84 denier, 85 denier, 86 denier, 87 denier, 88 denier, 89 denier, 90 denier, 91 denier, 92 denier, 93 denier, 94 denier, 95 denier, 96 denier, 97 denier, 98 denier, or 99 denier).

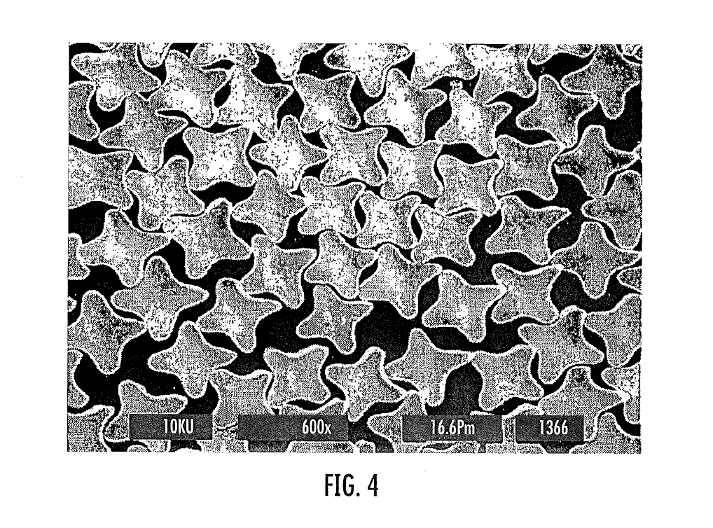

In some embodiments of the present invention, a continuous filament has non-round fiber cross-sections such as, for example, a star shaped cross section or a clover leaf cross section. The clover-leaf cross section also improves the fabric's smoothness and softness. Examples of these non-round fiber cross sections are illustrated in FIGS. 4 and 5. With non-round fiber cross sections, adjacent filaments form wicking channels along fiber surfaces to promote and enhance moisture transport away from contact with a person's skin. Thus, moisture more quickly evaporates and dries from the fabric surface, reducing the amount of moisture contacting the skin. The wicking channels also help the user to maintain body temperature by reducing excess sweating. The capillary action of fabrics according to embodiments of the present invention further serves to cause die-off of bacteria and other pathogens on the fabric. Moisture wicking reduces the overall moisture in the fabric which, therefore, helps dry out bacterial cells (and other pathogen cells) on the fabric, causing them to more readily die off.

In some embodiments of the present invention, nylon is used because it has one of the highest moisture regains of any synthetic fiber. Nylon absorbs moisture, and aids in wicking and evaporation. Polyester can also be used, according to some embodiments of the present invention, particularly if a durable auxiliary hydrophilic treatment is applied as a post finish.

In some embodiments, the fabric may also contain a soil-release topical finish. Thus, the fabric is able to release stains associated with skin antibiotic creams and ointments, as well as other stain-causing agents.

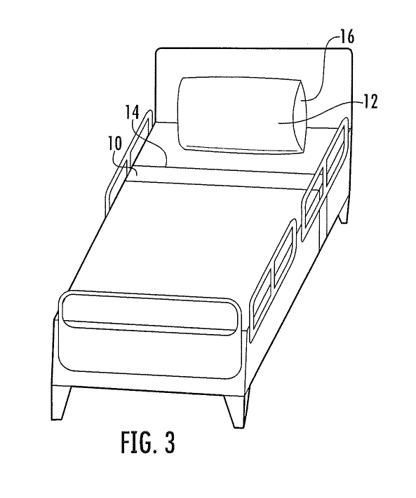

As seen in FIG. 3, a sheet 10 (i.e., a bottom sheet and/or a top sheet) fitted to a bed is made with a woven fabric having warp yarns and filling yarns woven to provide a smooth, identical fabric surface on both sides and sized to cover a bed. The sheet(s) 10 may have hems 14 to prevent raveling of the woven fabric. In some embodiments of the present invention, one of the warp or filling yarns is at least 40% by weight of the fabric of continuous filament polymer, and the other of the warp and filling yarns is 60% or less by weight of the fabric of continuous filament polymer having non-round filament cross sections. In some embodiments of the present invention, one of the warp or filling yarns is at least 40% by weight of the fabric of continuous filament nylon, and the other of the warp and filling yarns is 60% or less by weight of the fabric of continuous filament polyester or nylon having non-round filament cross sections.

In some embodiments of the present invention, an antimicrobial substance may be topically applied or inherently available in the fabric. In some embodiments, an antimicrobial substance such as AEGIS Microbe Shield, manufactured by Microban International (Huntersville, N.C.), is topically applied to the woven fabric in a standard textile finishing operation. This antimicrobial is effective against the following common microbes: Escherichia Coli, Staphylococcus Aureus, Staphylococcus Epidermidis, Pseudomonas Aeruginosa. The antimicrobial substance may also prevent odors in the fabric.

Also, as seen in FIG. 3, a pillowcase 12 is made with a woven fabric as described above. The pillowcase 12 is sewn to form a pocket to encase a pillow with an opening 16 on one end to enable insertion of a pillow therein. In some embodiments of the present invention, one of the warp or filling yarns is at least 40% by weight of the fabric of continuous filament polymer, and the other of the warp or filling yarns is 60% or less by weight of the fabric of continuous filament polymer having non-round filament cross sections. In some embodiments of the present invention, one of the warp or filling yarns is at least 40% by weight of the fabric of continuous filament nylon, and the other of the warp and filling yarns is 60% or less by weight of the fabric of continuous filament polyester or nylon having non-round filament cross sections. In some embodiments of the present invention, an antimicrobial substance is topically applied or inherently available in the fabric.

Fabrics according to embodiments of the present invention have high moisture regain and excellent moisture transport. Nylon, with one of the highest moisture regains of any synthetic fiber, absorbs moisture and aids in wicking and evaporation. Non-round fiber cross sections create channels along fiber surfaces to promote and enhance moisture transport away from contact with the skin. Moisture more quickly evaporates and dries, and thereby reduces the amount of wetness next to a person's skin. As such, fabrics according to embodiments of the present invention help a patient or other user maintain body temperature by reducing excess sweating. In some embodiments of the present invention, the fabric is 100% dry after 1 hour.

Fabrics according to embodiments of the present invention have very low friction with the skin of a patient. Continuous-filament yarns have no short fibers extending beyond the fabric's planar surface to create friction and to irritate sensitive skin. A smooth fabric surface accentuates this effect. In some embodiments of the present invention, the fabric has an average geometric roughness of less than about 1.7 microns as measured by the Kawabata Evaluation System FB4 Surface Tester.

Fabrics according to embodiments of the present invention have a good degree of stretch and recovery. Such fabrics help bed sheets to fit tighter and thereby reduce wrinkling that causes skin irritation. Such fabrics also better conform to the body and reduce the shear forces on sensitive skin. In some embodiments of the present invention, the fabric is finished to produce a fabric with an elongation greater than about 30% as measured by ASTM D5034-95.

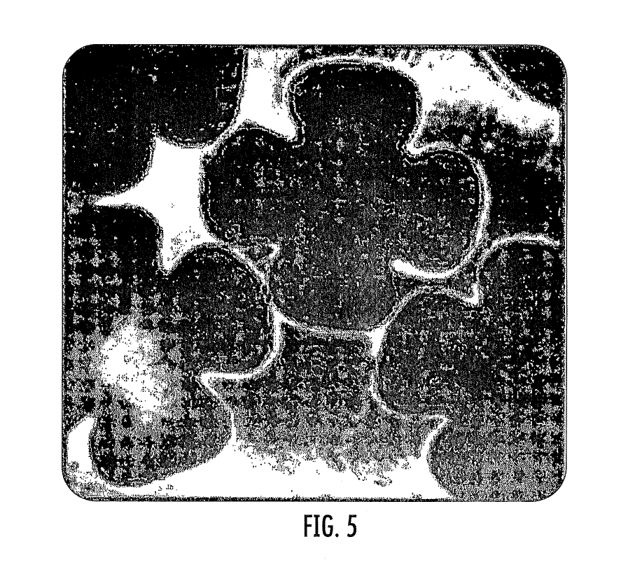

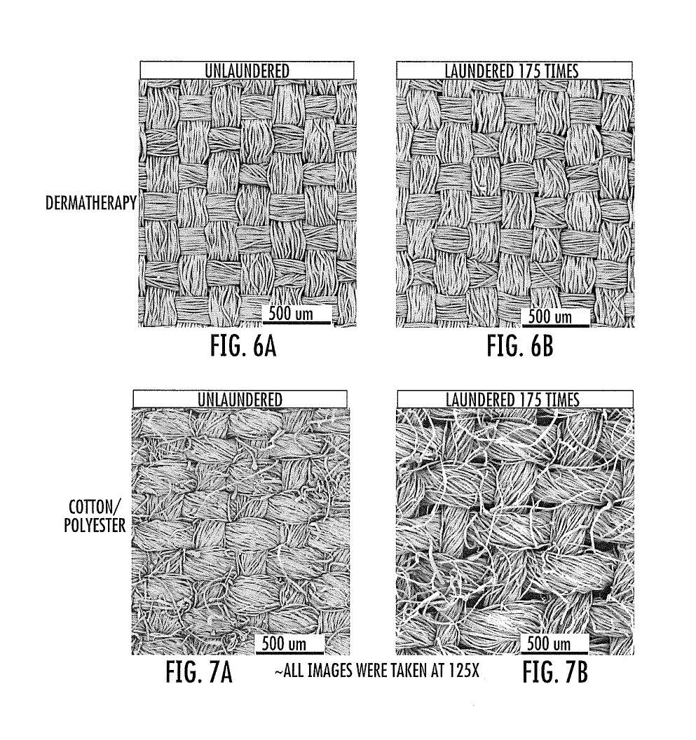

Fabrics according to embodiments of the present invention have durability to extended laundering and drying. Fabrics, according to embodiments of the present invention, will not lose fibers during laundering (in comparison with conventional cotton blends), and are not afflicted with fiber pills that further irritate skin. For example, FIG. 6A is a photomicrograph of a fabric according to some embodiments of the present invention. FIG. 6B is a photomicrograph of the fabric of FIG. 6A after being laundered one hundred seventy five (175) times. FIG. 6B demonstrates that fabric according to embodiments of the present invention is unaffected by extended laundering, even after one hundred seventy five (175) actual wash/dry cycles under hospital-laundry conditions.

In contrast, FIG. 7A is a photomicrograph of a conventional cotton/polyester hospital bedding fabric, and FIG. 7B is a photomicrograph of the cotton/polyester fabric of FIG. 7A after being laundered one hundred seventy five (175) times. As clearly illustrated in FIG. 7B, the conventional cotton/polyester fabric is substantially degraded as a result of extended laundering. Moreover, conventional hospital cotton/polyester fabrics lose substantial weight and most of their tensile strength during extended laundering.

Fabrics according to embodiments of the present invention are able to withstand high wash temperatures and the use of harsh detergents, and are able to release stains associated with skin antibiotic creams and ointments. Moreover, fabrics according to embodiments of the present invention have shown antibacterial efficacy against the survival of S. aureus, fungus, and molds on the fabric surface. In some embodiments of the present invention, the fabric has an antimicrobial efficacy against E. Coli, Staph. Epidermidis, and P. Aeruginosa of at least about 99.4% per AATCC 100.

Fabrics according to embodiments of the present invention have resistance to odors.

The data for fabrics woven with the yarns, such as illustrated in FIGS. 4 and 5, are compared with typical 55/45 polyester/cotton and 100% cotton bedding fabrics in Table I below.

TABLE 1

| | Present Invention | | | | Conventional Sheets | |
|---|---|---|---|---|---|---|
| | | Warp Yarn | | | | |
| | | 40/34 | 40/34 | 70/48 | 55/45 | 100% |
| | | 7z Nylon | 7z Nylon | Textured nylon | Poly/cotton | Cotton |
| | | Fill Yarn | | | | |
| | | 75/48 | 70/72 | 75/36 | 55/45 | 100% |
| | | Text. Poly. | Text. Nylon | Text. Poly. | Poly/cotton | Cotton |
| Fabric Weight | Osy | 2.53 | 2.51 | 2.43 | 3.72 | 3.44 |
| Yarns per Inch, warp | Epi | 180 | 173 | 102 | 111 | 113 |
| Yarns per Inch, fill | Ppi | 110 | 107 | 104 | 79 | 84 |
| Avg. Elongation | % | 37.6 | 37.4 | 39.6 | 17.2 | 12.8 |
| Air Permeability | cfm/ft² | 10.7 | 9.2 | 30.5 | 39.5 | 39.4 |
| Circular Bend | N | 0.7 | 0.6 | 0.3 | 0.9 | 0.7 |
| Thermal Insulation Value | Clo | 0.49 | 0.50 | n/a | 0.53 | 0.55 |
| Soil Release to Oily Stains | | 3.0 | 2.5 | 5.0 | 3.5 | 3.0 |
| Coefficient of Friction, warp | | 0.53 | 0.35 | 0.22 | 1.10 | 1.00 |
| Coefficient of Friction, fill | | 0.50 | 0.56 | 0.23 | 1.08 | 1.03 |
| Avg. Surface Roughness | M | 1.4 | 1.1 | 1.5 | 3.7 | 2.3 |
| Fabric Dryness after 1 hour | % | 100% | 100% | 100% | 51% | 52% |

Fabric according to embodiments of the present invention, referred to as DermaTherapy® bedding fabric S/66514, was tested to determine the weight loss when subjected to repeated launderings. The standard Polyester/Cotton blend hospital sheet was also tested to evaluate for weight loss and was compared with DermaTherapy® S/66514. DermaTherapy® fabric S/66514 and Polyester/Cotton blend hospital sheet samples were cut and weighed—three 2″×6″ samples of each. The test specimens were washed in a Laundrometer using the AATCC 61 test method which mimics several washings in one cycle. For this testing, 5A wash settings were used, that represents 5 washes in one cycle. After the wash cycle, the specimens were transferred to the dryer. After the dryer cycle, the test specimens were reconditioned for 1 hour. The specimens were then weighed and the percent weight loss was calculated using the following calculations:

$$\% Weight Loss = \frac{Original - Final}{Original} \times 100$$

Weight loss results for DermaTherapy® bedding fabric S/66514 and for standard Polyester/Cotton blend hospital sheet are summarized in Table 2 below.

TABLE 2

| | DermaTherapy ® | Polyester/Cotton |
|---|---|---|
| Number of | Style/66514 | Hospital Sheet |
| Launderings | Weight Loss % | Weight Loss % |
| 1x | 0% | 1.51% |
| 25X | 0% | 3.92% |
| 50X | 0% | 6.25% |
| 75X | 0% | 7.26% |
| 100X | 0% | 8.24% |

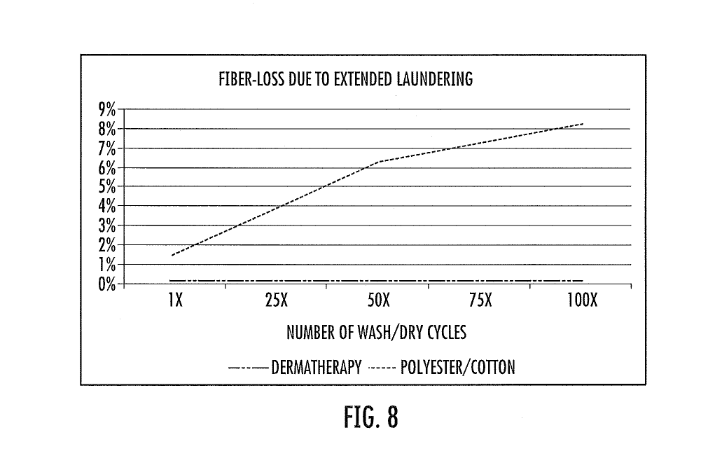

As clearly illustrated in Table 2, fabric according to embodiments of the present invention did not lose any fibers as a result of laundering and, thus, had no weight loss. In contrast, the conventional Polyester/Cotton blend hospital sheet exhibited significant weight loss. This is graphically illustrated in FIG. 8, also.

Fabrics according to embodiments of the present invention are capable of retaining antimicrobial substances applied thereto much longer than conventional hospital sheet fabrics. The antimicrobial substance is applied as a surface treatment (not a coating) to the fabrics according to the embodiments, where the active ingredient is molecularly bound to the fiber surfaces. The application process involves processing the fabric through a bath of chemicals, followed by a series of pad/squeeze rolls to remove excess chemicals leaving a precise amount of chemicals on the fabric, and finally through a drying process where the molecular bonding takes place. The inactive ingredient (water) is driven off during the drying process and does not otherwise remain on or in the fabric. After processing, the fabric is clean and free of any chemical residues. The active ingredient in AEGIS forms a colorless, odorless, positively charged polymer that molecularly bonds to the treated surface. It can be thought of as a surface layer of electrically charged swords. When a microorganism comes in contact with the treated surface, the C-18 molecular sword punctures the cell membrane and the positive electrical charge disrupts the cell wall. Since nothing is transferred to the now dead cell, the antimicrobial does not lose strength and the molecule is available for the next cell to contact it. The antimicrobial substance is an “organofunctional silane” with a quaternary ammonium component. The silane component permanently attaches the molecule to the fabric surface. The cured antimicrobial does not volatilize, dissipate, or leach onto other surfaces or into the environment. The chemistry polymerizes where it is applied and forms a permanent bond that typically lasts for the life of the treated surface. Therefore, fabrics according to embodiments of the present invention retain antimicrobial substances applied thereto even after extended laundering.

In providing a therapeutic environment for bedridden patients, appropriate moisture conditions should be maintained to prevent or reduce ulceration. An overly dry condition may lead to a skin more vulnerable to cracking. Conversely, excessive hydration, for example because of incontinence and/or perspiration, may cause skin maceration and lower the tissue tolerance to shear stress and friction. Moisture from sweating or incontinence hydrates the skin, dissolving the molecular collagen crosslinks of the dermis, and softening the stratum corneum (maceration). Increased perspiration is particularly relevant to pressure ulcer risk because moisture on the skin surface can reduce the skin's resilience and increase the COF (both static and kinetic) of the skin, making the skin more prone to pressure, shear stresses and friction. Skin maceration reduces stiffness, threatening the near complete loss of connective tissue strength and the erosion of the dermis under the action of shear forces. Another result of skin hydration is the rapid increase of epidermal friction, which promotes adhesion of the skin to the support surface—covered by bed linens—and increases shear, easy sloughing, and ulceration.

The micro-fibers in fabrics according to embodiments of the present invention form tiny channels that rapidly wick away moisture from the surface of the bedding when the fabric becomes wet from perspiration, a draining wound, incontinence from stool or urine, etc. Fabrics according to embodiments of the present invention are designed to be smooth and soft such that friction between the skin and fabric surface is greatly reduced.

Bed linens and other support surface covers, according to embodiments of the present invention, play an important role in moderating liquid and moisture to maintain a healthier microclimate near the skin surface. It is known that cotton-blend fabrics are not very effective in managing moisture and wetness. For example, it is known that the kinetic COF between skin on completely wet cotton-polyester fabrics is more than twice the value for natural skin on a dry textile surface. Fabrics according to embodiments of the present invention are engineered to maximize moisture wicking and drying, while minimizing frictional properties of hospital bed linens and other fabric products. Moreover, fabrics according to embodiments of the present invention reduce friction with the skin of a person without requiring that the fibers of the fabric be treated with low friction material.

Any surface that comes into contact with a person's skin has the potential to alter the microclimate between the skin and the fabric surface by changing the rate of moisture evaporation—and thus the drying rate—and the rate at which heat dissipates from the skin. The overall effect of a support surface on microclimate is dependent on numerous factors, including the nature of the support surface itself, i.e., what materials the support surface are made from, how fabric materials are conformed to cover the support surface, and how the materials inherently affect moisture, friction and shear between the skin and the support surface.

Fabrics according to embodiments of the present invention are designed of special micro-denier fibers and yarns, woven into a fabric that facilitates rapid wicking and drying through moisture evaporation in order to prevent heat and moisture accumulation at the skin-fabric interface. Its effectiveness in doing so can be measured in terms of water loss from the fabric surface. The evaporation of 1 Kg of water from the skin at a support surface has been found to remove 580 Kcal of heat from the body through the “latent heat of vaporization.” Thus, for an average person with 1.8 m² of skin surface, a water loss of approximately 26.7 g/m²/hour is needed to maintain a physiologic water balance and maintain a normal cooling power of 27.9 Kcal/hour. At a wicking rate of 1.00-1.75 inches per minute and a dryness of greater than 90% after 45 minutes, fabrics according to embodiments of the present invention have been shown to provide a water loss of approximately 46.0 g/m²/hour, while achieving complete dryness in 55 minutes under normal room temperature and humidity (e.g., between about 65° F. and about 79° F., and between about 40% RH and about 60% RH; although other ranges are possible within the meaning of normal room temperature and humidity). This more than exceeds the water loss of 26.7 g/m²/hour needed to maintain a physiologic water balance, and thereby more effectively controls skin temperature and moisture.

By comparison, in tests performed by the Applicant, conventional cotton-blend hospital fabrics have been shown to achieve a water loss of 61.8 g/m²/hour, but still retain 17.5 g/m² of water after one hour. These tests were performed and verified according to the standard test method, INDA IST 10.1 (EDANA 10.4-02) Wicking Rate, “Standard Test Method for Wicking Rate.” That is, conventional cotton-blend fabrics hold more water, but release the water much more slowly through evaporation. Fabrics according to embodiments of the present invention are clearly superior to conventional cotton-blend fabrics in efficiently managing moisture to maintain a physiologic water balance. As such, fabrics according to embodiments of the present invention positively affect the moisture-related conditions impacting the development of pressure wounds in patients.

Pressure, friction and shear are all intimately linked. Pressure on soft tissues, especially when over a bony prominence of a person, may cause some degree of shear through tissue distortion. Friction contributes to the development of shear stresses by tending to keep the skin in place against a support surface while the rest of the patient's body moves on the support surface, for example, towards the foot of a bed. The relative movement of a person's skin and the underlying tissues causes shear stresses to develop in the soft tissues overlying bony prominences such as, for example, the sacrum. Shear stresses may act in conjunction with pressure to produce the damage and ischemia of the skin and deeper tissues that result in pressure ulcers. The mechanisms involved include distortion of tissues, pinching and occlusion of capillaries crossing tissue planes, reductions in blood flow, and physical disruption of tissues or blood vessels.

The three most commonly used pressure ulcer risk scales (Braden, Norton and Waterlow) all recognize moisture or incontinence as a risk factor for pressure ulcers. The Braden scale specifically evaluates friction and shear, based on the level of assistance required to move on a surface, frequency of sliding in a bed or chair, and the presence of spasticity, contractures or agitation that cause friction. The significance of friction in the context of pressure ulcers lies mainly in its contribution to the production of shear stresses. When the tangential force applied by friction at the skin surface is larger than the perpendicular force (pressure), or when a small amount of pressure with a large tangential force is applied to the skin, abrasions, superficial ulceration or blistering may occur. If the skin is already irritated or inflamed, e.g., by maceration or incontinence-associated dermatitis, superficial damage due to friction may occur more easily. Friction applied to the skin surface may also cause shear stresses in deeper tissue layers such as muscle.

The surface smoothness of fabrics and the stiffness/flexibility of fibers and fabrics are important factors in determining the shear and friction experienced by patients. When a bed sheet covering a support surface is designed to allow movement over its foundation, slippage occurs between the bed sheet and the bed and not within the body tissue layers; thus, tension or stretching in the skin and blood vessel occlusion are decreased due to a lower COF.

The COF of fabrics against the skin of a person may be influenced by the following:
1) The nature of the fabric; e.g., rougher fabrics produce higher COF.
2) Skin moisture content and surface wetness, for example from perspiration or because of incontinence, which increase the COF.
3) Ambient humidity may increase skin moisture content or induce sweating and therefore increase COF.
   Bed-bound individuals experience prolonged pressure and shear/friction caused by the conventional textile products currently in use today that put them at risk for developing pressure ulcers. Specifically, this may occur through skin rubbing against the bed sheets; skin being pulled from repeated episodes of sliding down in the bed; heels sliding against the bed linens, and friction on the skin caused by repeated episodes of pulling up in the bed. Shear, accompanied by friction, are the proximate cause of skin damage in each case.

As skin moisture increases, the COF (both static and kinetic) increases between a person's skin and polyester/cotton textile materials. In fact, the COF for wet fabric on skin has been shown to be more than double the value for dry fabric on skin. Gerhardt studied how epidermal hydration affects friction between the skin and textile fabrics. Twenty two Caucasian participants, at an average age of 31.7 years with average BMI (body mass index) of 23.3, rubbed their inner forearm against a hospital fabric and a plate measuring force. Gerhardt found that COFs of skin against wet cotton-polyester fabric exceeded those of natural skin by a factor of more than two. (Gerhardt L C, Strassle, Lenz A, Spencer N D, Derler S. Influence of epidermal hydration on the friction of human skin against textiles. J. R. Soc. Interface. 2008; 5:1317-1328.)

The COF (both static and kinetic) between a conventional dry cotton-polyester hospital fabric and skin is typically about 0.43, while the COF for the same fabric in a wet condition against skin is typically in a range from about 0.8 to about 1.0. The fact that the COF of conventional cotton-blend hospital fabrics dramatically increases in the presence of moisture illustrates an important deficiency for patients at-risk of developing pressure ulcers. It should be noted that fabrics according to embodiments of the present invention resolve this tendency towards higher friction coefficients through the use of non-hygroscopic continuous-filament synthetic yarns woven into a smooth plain weave. Fabric-to-fabric COF measurements for both a fabric according to embodiments of the present invention (labeled DermaTherapy®) and conventional cotton-blend bedding fabrics are shown in Table 3 below.

TABLE 3

| | COF - Dry | COF - Wet |
|---|---|---|
| DermaTherapy ® fitted | 0.30 | 0.22 |
| sheet | | |
| Cotton/polyester fitted | 0.28 | 0.42 |
| sheet | | |

Fabrics according to embodiments of the present invention have a kinetic COF that does not exceed 0.35 under both dry and wet conditions. As illustrated in Table 3, fabrics, according to embodiments of the present invention, have a kinetic COF-Dry of 0.30 and a kinetic COF-Wet of 0.22. In sharp contrast, conventional cotton/polyester fabrics have a kinetic COF-Dry of 0.28 and a kinetic COF-Wet of 0.42. As such, this overcomes the inherent deficiency of conventional cotton-blend hospital bedding fabrics of increased COF when wet, while maintaining a consistently low overall COF needed to minimize skin abrasion.

### Clinical Trial

The primary objective of the clinical trial was to evaluate the therapeutic benefits of bed linens, underpads, and patient gowns, made with DermaTherapy® fabrics, according to embodiments of the present invention, to minimize friction, shear and pressure damage to the skin and thereby reduce and/or prevent pressure wounds. The trial was carried out at the Medical Renal Ward at Moses H. Cone Memorial Hospital in Greensboro, N.C. The patient population in the Medical Renal Ward was chosen because it had multiple co-morbidities in addition to chronic renal failure and had been identified as high risk for pressure wound development. All inpatients admitted or transferred to the Renal Ward who met the entrance criteria were asked to consent to be part of the trial. Male or female patients of any race, admitted to the Medical Renal Unit for greater than two consecutive days, were included. Patients who were placed on any specialty beds (e.g., pressure-reduction beds, bariatric beds) upon admission were excluded. Certain information was collected at admission for all the patients who participated in the study. This data, summarized below, was collected to perform a fair comparison between the subjects.
Patient demographic data, including weight, gender, age, and the medical diagnosis of each patient.
Co-morbidities, such as hypertension, anemia, diabetes or kidney disease.
Albumin level, a basic screening tool in monitoring protein levels. Albumin levels are a gross indicator of nutritional status and fluid balance. This test was done on all the patients at the time of admission to determine the severity of their protein deficiency. The extent to which albumin levels are below normal predict the risk of pressure ulcer formation. Albumin levels greater than 3.6 g/dl are considered to be normal.
Braden Scores, a widely used and validated pressure ulcer risk assessment tool available to assess patient risk of developing pressure ulcers. The Braden scale is composed of: six subscales: sensory perception (the ability to respond to pressure related discomfort), moisture (the degree to which skin is exposed to perspiration, wound drainage, urine, and stool), nutrition (the usual food intake pattern), mobility (the ability to change and control body position), friction and shear (the presence of friction and shear forces—e.g. the degree to which the skin slides against a fixed surface such as bed or chair), and activity (the degree of physical activity). The score in each category indicates the risk potential, where 1 indicates the greatest risk and 4 indicates the least risk. Patients with Total Braden Scores of 15 to 18 are considered “at risk” for developing pressure wounds. In this trial, the Braden scale score was determined at admission.

Skin assessments were made each day on each patient in the trial. A checklist was used to record and monitor pressure wound information, such as the wound locations, stages, colors, and inflammation/conditions. The trial was conducted in three sessions: eight weeks on Control items (98 patients); then eight weeks on DermaTherapy® items (153 patients); followed by eight weeks on Control items (56 patients).

The Control items included standard hospital cotton-blend bedding (flat top sheet, fitted bottom sheet, and pillowcase), underpads, and patient gowns. The DermaTherapy® items included a flat top sheet, fitted bottom sheet, a pillow case, underpad and patient gown, all made with the DermaTherapy® fabric. The DermaTherapy® underpad was identical to the standard hospital underpad with the exception of the top surface fabric. That is, the surface fabric—the layer closest to the patients' skin—was comprised of the DermaTherapy® fabric. The inner “soaker” layer and moisture barrier used in the DermaTherapy® underpad were the same as used in the standard Control underpad.

The objective of the trial was to evaluate and compare the Control and DermaTherapy® items in reducing friction, shear, and pressure damage to the skin. In turn, reductions in maceration and skin fold breakdown were evaluated, where the bedding, underpad, and patient gown helped protect moist skin folds caused by sweat and incontinence.

Data obtained from this study was analyzed using the t-test statistical method assuming equal variances and one-tailed distributions. The t-test was used to determine whether two sample means are equal. The t-test assumes sampling without any selection bias. Therefore, in this study, study participants were assigned to either the Control or DermaTherapy® session so that two sessions do not have any systematic difference except for the treatment applied. Patients were not told which bed linens or gowns they were using during the trial. Statistical significance was indicated when p<0.05; that is, there was a 95% chance or greater that the differences in the means, or averages, were not due to chance.

A total of 307 patients participated in the trial. Statistical significance between the Control and DermaTherapy® sessions was determined using the t-test. All data in the discussion that follows are expressed as means (i.e., averages). In the data comparisons, the results from the two Control sessions were combined and compared with DermaTherapy® session results. Intersession comparisons were also made.

Tables 4, 5, and 6 summarize comparative analyses of patients who participated in the Control and DermaTherapy® sessions. Very few differences were seen between Controls and DermaTherapy® at Admittance. Out of 307 patients, 89 were males and 101 were females with mean age of 63.2 years for the Control patients and 62.5 years for the DermaTherapy® patients. The proportion of men in the DermaTherapy® session was 13% higher than in the Control sessions. Albumin levels for patients in this study averaged 2.80 to 2.84, well below normal albumin levels of 3.6 and higher.

TABLE 4
Admittance data analysis - Demographics of Control Patients
versus DermaTherapy ® Patients

| | Controls | DermaTherapy ® | % | |
|---|---|---|---|---|
| | n = 154 | n = 153 | Difference | p-value |
| Weight (Pounds) | 176.4 | 173.9 | −1.4% | 0.3241 |
| Age (Years) | 63.2 | 62.5 | −1.1% | 0.2277 |
| Albumin Level | 2.84 | 2.80 | −1.4% | 0.3236 |
| No. Co-morbidities | 4.6 | 4.29 | −6.7% | 0.1054 |
| Gender, | 38/62 | 51/49 | | 0.0149 |
| % Male/% Female | | | | |

The results from statistical analysis shown in Table 5 indicate very few differences in co-morbidities of patients at Admittance between Control patients and DermaTherapy® patients.

TABLE 5
Co-morbidity data analysis - Percentage of
Patients with Co-Morbidities at Admittance

| | Controls | DermaTherapy ® | |
|---|---|---|---|
| | n = 154 | n = 153 | p-value |
| Hypertension | 76.0% | 70.6% | 0.1439 |
| Anemia | 65.6% | 51.0% | 0.0047 |
| Diabetes | 53.2% | 49.0% | 0.2302 |
| Renal Failure | 52.0% | 44.4% | 0.0947 |
| Kidney Disease | 34.4% | 32.7% | 0.3742 |
| Atherosclerosis | 32.5% | 31.4% | 0.3137 |
| Pulmonary | 28.6% | 26.1% | 0.3173 |
| Heart Failure | 20.8% | 15.0% | 0.0952 |
| CVA | 17.5% | 17.6% | 0.4895 |
| Infectious Disease | 13.0% | 17.6% | 0.1292 |
| Neoplasm | 14.3% | 16.3% | 0.3093 |
| Drugs/Alcohol | 9.1% | 16.3% | 0.0284 |
| Thrombo/Plebitis | 7.8% | 9.2% | 0.3352 |
| Myocard. Infarction | 6.5% | 9.8% | 0.1452 |

Differences in Braden component scores and total Braden scores between Control patients and DermaTherapy® patients at Admittance were also very small. Patients with Braden Scores of 15 to 18 are considered “at risk” for developing pressure wounds. As shown in Table 6, Braden Scores for patients in this study averaged 17.1 in both Control Sessions and DermaTherapy® Session.

TABLE 6
Braden data analysis - Braden Scores for Control Patients
versus DermaTherapy ® Patients

| | Controls | DermaTherapy ® | % | |
|---|---|---|---|---|
| | n = 154 | n = 153 | Difference | p-value |
| Perception | 3.1942 | 3.1517 | 1.3% | 0.2764 |
| Moisture | 3.4964 | 3.5586 | 1.8% | 0.1855 |
| Activity | 2.6547 | 2.5586 | −3.6% | 0.2058 |
| Mobility | 2.9784 | 2.9379 | −1.4% | 0.3061 |
| Nutrition | 2.4892 | 2.5310 | 1.7% | 0.3105 |
| Friction | 2.2662 | 2.3310 | 2.9% | 0.1814 |
| Total Braden | 17.0791 | 17.069 | −0.1% | 0.4883 |
| Scores | | | | |

All of the patients in the trial exhibited some risk of developing pressure wounds at Admittance. The collective data analysis of patients at Admittance indicates a good homogeneous mix, without undue influence on the subsequent treatment phase.

A statistical analysis was performed on the wound assessment data obtained for each patient on a daily basis. Table 7 below shows comparisons between the Control patients and DermaTherapy® patients in terms of the average number of wounds per patient. At Admittance, wound data analysis indicates 19.5% more wounds were evident for those patients using DermaTherapy® items; however, the difference between the Control and DermaTherapy® patients was not found to be statistically significant (p=0.259). By the end of the 24-week trial period, those patients using DermaTherapy® bedding and gowns developed 61.7% fewer wounds than those patients using the Control items (p=0.014). Those patients using DermaTherapy® also experienced a higher resolution of their wounds. “Wounds Resolved During Stay” indicates the average number of wounds that were either evident at admittance or developed during their stay, but were not evident at discharge from the Medical Renal Unit. In the study, for those patients using DermaTherapy® items, 91.4% more wounds were resolved than for those patients using the Control items (p=0.070). Patients using DermaTherapy® items also exhibited 39.6% fewer wounds at Discharge from the Renal Unit than those patients in the Control group (p=0.0315). Comparisons were also made within the Control and DermaTherapy® groups. The average number of wounds per patient in the Control group increased 41% between Admittance and Discharge, while the average number of wounds per patient In the DermaTherapy® group declined by 29%.

TABLE 7
Wound Data Analysis Average Number of Wounds per Patient

| | Controls | DermaTherapy ® | % | |
|---|---|---|---|---|
| | n = 154 | n = 153 | Difference | p-value |
| Wounds at | 0.208 | 0.248 | 19.5% | 0.259 |
| Admittance | | | | |
| Wounds Developed | 0.136 | 0.052 | −61.7% | 0.014 |
| During Stay | | | | |
| Wounds Resolved | 0.058 | 0.111 | 91.4% | 0.070 |
| During Stay | | | | |
| Wounds at | 0.279 | 0.177 | −36.6% | 0.050 |
| Discharge | | | | |

The results from this analysis were than confirmed, by the outcomes obtained by normalizing the wound data for patients' length of stay. The data obtained was normalized to show the average number of pressure wounds per 1,000 patient days.

In a comparative analysis, shown in Table 8 below, it was found that the results obtained per-1,000 patient days were in line with results computed on a per-patient basis. The average wounds per 1,000 patient days at Admittance were 34% higher for DermaTherapy® patients; however, the difference was not statistically significant (p=0.259). The development of wounds per 1,000 patient days was 57% less with DermaTherapy® as compared to Control patients (p=0.014). With DermaTherapy®, the average number of wounds per 1,000 patient days was 113% lower than the rate for Control patients (p=0.070). Wounds at discharge were found to be significantly lower, at 32%, than found with the Control patients (p=0.032). Again, comparisons were made within the Control and DermaTherapy® groups. The average number of wounds per 1,000 patient-days in the Control group increased 40% between Admittance and Discharge, while the average number of wounds per patient in the DermaTherapy® group declined by 29% between Admittance and Discharge.

TABLE 8
Wound Data Analysis Average Number
of Wounds per 1,000 Patient-Days

| | Controls | DermaTherapy ® | % | |
|---|---|---|---|---|
| | n = 154 | n = 153 | Difference | p-value |
| Wounds at | 34.9 | 46.8 | 34.2% | 0.259 |
| Admittance | | | | |
| Wounds Developed | 22.9 | 9.9 | −56.8% | 0.014 |
| During Stay | | | | |
| Wounds Resolved | 9.8 | 20.9 | 113.3% | 0.070 |
| During Stay | | | | |
| Wounds at | 49.0 | 33.3 | −32.0 | 0.032 |
| Discharge | | | | |

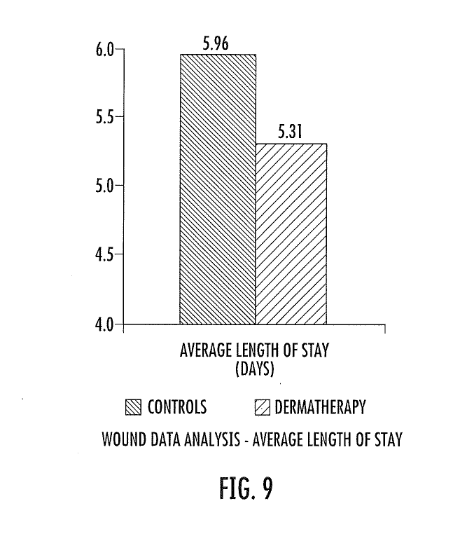

In addition to the improvements in wound development, resolution, and incidence at Discharge, the average length of stay for patients in the Medical Renal Unit using DermaTherapy® were reduced by 11% (0.65 days), as compared with those patients using the Control items (p=0.705), as illustrated in FIG. 9.

For those patients using the DermaTherapy® bed linens and gowns, statistically significant differences were demonstrated. For example, a 62% reduction in the average number of wounds per patient developed during patients' stay on the Renal Unit (p=0.014) and a 40% reduction in wounds at Discharge (p=0.0315) was achieved. Statistically strong improvements were also seen: a 90% increase in the average number of wounds resolved (p=0.070) and an 11% reduction in patients' average length of stay (p=0.071). This data provides clear evidence that state-of-the-art synthetic textile materials, according to embodiments of the present invention, have the potential to significantly contribute to better healthcare outcomes and lower overall system costs. Fewer wounds developed during hospital stays lead to greater comfort and less suffering by patients, as well as lower health care costs at hospitals, rehabilitation facilities, and nursing homes.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A fabric comprising a woven fabric having continuous filament warp yarns and continuous filament filling yarns, wherein the warp yarns are about 30 denier to 100 denier, and the filling yarns are about 30 denier to 100 denier, wherein the warp yarns and filling yarns are woven together in a plain weave to provide a pill resistant fabric having identical surfaces on both sides thereof, and wherein each surface has a coefficient of friction that does not exceed 0.35 under both dry and wet surface conditions.

2. The fabric of claim 1, wherein each surface has a coefficient of friction that does not exceed 0.30 under both dry and wet surface conditions.

3. The fabric of claim 1, wherein each surface has a coefficient of friction that does not exceed 0.30 under dry conditions and a coefficient of friction that does not exceed 0.22 under wet conditions.

4. The fabric of claim 1, wherein the fabric has a wicking rate of between about 1.00 and 1.75 inches per minute and a percent (%) dryness after 45 minutes of 90%.

5. The fabric of claim 1, wherein one of the warp or filling yarns comprises 100% continuous filament polymer having round filament cross sections and making up at least 40% by weight of the fabric, and the other of the warp or filling yarns comprises continuous filament polymer having non-round filament cross sections and making up the remainder of the weight of the fabric.

6. The fabric of claim 1, wherein one of the warp or filling yarns comprises 100% continuous filament nylon having round filament cross sections and making up at least 40% by weight of the fabric, and the other of the warp or filling yarns comprises continuous filament polyester having non-round filament cross sections or nylon having non-round filament cross sections and making up the remainder of the weight of the fabric.

7. The fabric of claim 6, wherein the warp yarns are 100% nylon and the filling yarns are 100% polyester.

8. The fabric of claim 5, wherein the warp or filling yarns with non-round filament cross sections are configured such that adjacent filaments form wicking channels.

9. The fabric of claim 6, wherein the warp and filling yarns are a 70 denier, 200 filament, textured, continuous filament polyester yarn.

10. The fabric of claim 5, wherein the non-round filament cross sections are star shaped cross sections.

11. The fabric of claim 5, wherein the non-round filament cross sections are clover leaf cross sections.

12. The fabric of claim 1, further comprising a soil-release topical finish.

13. The fabric of claim 1, wherein an antimicrobial substance is topically applied or inherently available in the fabric.

14. The fabric of claim 1, wherein the fabric has zero percent (0%) weight loss after one hundred (100) launderings in accordance with the AATCC 61 test method.

15. Use of the fabric of claim 1 in the manufacture of bed sheets for reducing the number of patient pressure wounds developed within a medical facility.

16. Use of the fabric of claim 1 in the manufacture of bed sheets for reducing patient pressure wounds within a medical facility, wherein the average number of pressure wounds developed by patients in the medical facility per one thousand (1,000) patient days is reduced by more than half (e.g., 50%) for patients occupying beds fitted with the bed sheets.

17. Use of the fabric of claim 1 in the manufacture of bed sheets for reducing patient pressure wounds within a medical facility, wherein the average number of pressure wounds resolved per one thousand (1,000) patient days is increased by at least about twenty nine percent (29%) for patients occupying beds fitted with the bed sheets.

18. Use of the fabric of claim 1 in the manufacture of hospital gowns for reducing patient pressure wounds within a medical facility.

19. Use of the fabric of claim 1 in the manufacture of bed sheets and hospital gowns for reducing patient pressure wounds within a medical facility, wherein the average number of pressure wounds developed by patients in the medical facility per one thousand (1,000) patient days is reduced by more than half (e.g., 50%) for the patients occupying beds fitted with the bed sheets and wearing the hospital gowns.

20. Use of the fabric of claim 1 in the manufacture of bed sheets and hospital gowns for reducing patient pressure wounds within a medical facility, wherein the average number of pressure wounds resolved per one thousand (1,000) patient days is increased by at least about twenty nine percent (29%) for patients occupying beds fitted with the bed sheets and wearing the hospital gowns.

21. A method of reducing the number of pressure wounds developed by a patient within a medical facility, comprising:
fitting a bed within the medical facility with clean sheets, wherein each sheet comprises a woven fabric having warp yarns and filling yarns woven together in a plain weave to provide a pill resistant fabric having identical surfaces on both sides thereof, and wherein each surface has a coefficient of friction that does not exceed 0.35 under both dry and wet surface conditions, then occupying the bed with the patient.

22. The method of claim 21, wherein each surface has a coefficient of friction that does not exceed 0.30 under both dry and wet surface conditions.

23. The method of claim 21, wherein each surface has a coefficient of friction that does not exceed 0.30 under dry conditions and a coefficient of friction that does not exceed 0.22 under wet conditions.

24. The method of claim 21, wherein the fabric has a wicking rate of between about 1.00 and 1.75 inches per minute and a percent (%) dryness after 45 minutes of 90%.

25. The method of claim 21, wherein one of the warp or filling yarns comprises 100% continuous filament polymer having round filament cross sections and making up at least 40% by weight of the fabric, and the other of the warp or filling yarns comprises continuous filament polymer having non-round filament cross sections and making up the remainder of the weight of the fabric.

26. The method of claim 21, wherein one of the warp or filling yarns comprises 100% continuous filament nylon having round filament cross sections and making up at least 40% by weight of the fabric, and the other of the warp or filling yarns comprises continuous filament polyester having non-round filament cross sections or nylon having non-round filament cross sections and making up the remainder of the weight of the fabric.

27. The method of claim 26, wherein the warp yarns are 100% nylon and the filling yarns are 100% polyester.

28. The method of claim 25, wherein the warp or filling yarns with non-round filament cross sections are configured such that adjacent filaments form wicking channels.

29. The method of claim 26, wherein the warp and filling yarns are a 70 denier, 200 filament, textured, continuous filament polyester yarn.

30. The method of claim 25, wherein the non-round filament cross sections are star shaped cross sections.

31. The method of claim 25, wherein the non-round filament cross sections are clover leaf cross sections.

32. The method of claim 21, further comprising a soil-release topical finish.

33. The method of claim 21, wherein an antimicrobial substance is topically applied or inherently available in the fabric.

34. The method of claim 21, wherein the fabric has zero percent (0%) weight loss after one hundred (100) launderings in accordance with the AATCC 61 test method.

35. The method of claim 21, further comprising dressing the patient with a clean hospital gown, wherein the hospital gown comprises a woven fabric having warp yarns and filling yarns woven together in a plain weave to provide a pill resistant fabric having identical surfaces on both sides thereof, and wherein each surface has a coefficient of friction that does not exceed 0.35 under both dry and wet surface conditions.

36. A method of reducing the number of pressure wounds developed by a patient within a medical facility, comprising:
dressing the patient with a clean hospital gown, wherein the hospital gown comprises a woven fabric having warp yarns and filling yarns woven together in a plain weave to provide a pill resistant fabric having identical surfaces on both sides thereof, and wherein each surface has a coefficient of friction that does not exceed 0.30 under both dry and wet surface conditions;
fitting a bed with clean sheets, wherein each sheet comprises the woven fabric; and then
occupying the bed with the patient.

37. The method of claim 36, wherein each surface has a coefficient of friction that does not exceed 0.30 under dry conditions and a coefficient of friction that does not exceed 0.22 under wet conditions.

38. The method of claim 36, wherein the fabric has a wicking rate of between about 1.00 and 1.75 inches per minute and a percent (%) dryness after 45 minutes of 90%.

39. The method of claim 36, wherein one of the warp or filling yarns comprises 100% continuous filament polymer having round filament cross sections and making up at least 40% by weight of the fabric, and the other of the warp or filling yarns comprises continuous filament polymer having non-round filament cross sections and making up the remainder of the weight of the fabric.

40. The method of claim 36, wherein one of the warp or filling yarns comprises 100% continuous filament nylon having round filament cross sections and making up at least 40% by weight of the fabric, and the other of the warp or filling yarns comprises continuous filament polyester having non-round filament cross sections or nylon having non-round filament cross sections and making up the remainder of the weight of the fabric.

41. The method of claim 40, wherein the warp yarns are 100% nylon and the filling yarns are 100% polyester.

42. The method of claim 39, wherein the warp or filling yarns with non-round filament cross sections are configured such that adjacent filaments form wicking channels.

43. The method of claim 40, wherein the warp and filling yarns are a 70 denier, 200 filament, textured, continuous filament polyester yarn.

44. The method of claim 39, wherein the non-round filament cross sections are star shaped cross sections.

45. The method of claim 39, wherein the non-round filament cross sections are clover leaf cross sections.

46. The method of claim 36, further comprising a soil-release topical finish.

47. The method of claim 36, wherein an antimicrobial substance is topically applied or inherently available in the fabric.

48. The method of claim 36, wherein the fabric has zero percent (0%) weight loss after one hundred (100) launderings in accordance with the AATCC 61 test method.

49. A fabric comprising a woven fabric having continuous filament warp yarns and continuous filament filling yarns, wherein the warp yarns are about 30 denier to 100 denier, and the filling yarns are about 30 denier to 100 denier, wherein the warp yarns and filling yarns are woven together in a plain weave to provide a pill resistant fabric having identical surfaces on both sides thereof, and wherein each surface has a coefficient of friction that does not exceed 0.30 under both dry and wet surface conditions;
wherein the fabric has zero percent (0%) weight loss after one hundred (100) launderings in accordance with the AATCC 61 test method; and
wherein the fabric has a wicking rate of between about 1.00 and 1.75 inches per minute and a percent (%) dryness after 45 minutes of 90%.

50. The fabric of claim 49, wherein each surface has a coefficient of friction that does not exceed 0.30 under dry conditions and a coefficient of friction that does not exceed 0.22 under wet conditions.
